(19) United States
(12) Patent Application Publication
(10) Pub. No.: US 2006/0019933 A1
(43) Pub. Date: Jan. 26, 2006

(54) Process for preparing stabilized vitamin D

(76) Inventors: David Boardman, Norristown, PA (US); Shyam B. Karki, Lansdale, PA (US); Aquiles E. Leyes, Schwenksville, PA (US); Drazen Ostovic, Newbury Park, CA (US)

Correspondence Address: MERCK AND CO., INC, P O BOX 2000, RAHWAY, NJ, 07065-0907, US

(21) Appl. No.: 11/176,610

(22) Filed: Jul. 7, 2005

Related U.S. Application Data
(60) Provisional application No. 60/590,173, filed on Jul. 22, 2004.

(51) Int. Cl. A61K 31/59

(52) U.S. Cl. 514/167

(57) ABSTRACT

This invention is related to a process for preparing a stabilized form of vitamin D which comprises the steps of: a) dissolving vitamin D in a solution that contains water and at least one surfactant to produce a mixture; b) spraying the mixture onto an inert carrier to produce a wet mass; and c) drying the wet mass obtain the stabilized form of vitamin D.

### BACKGROUND OF THE INVENTION

Direct addition of low concentrations of neat vitamin D to solid dosage formulations suffers from two significant drawbacks. One of the challenges is the extremely low thermal stability of the neat form of vitamin D, particularly in amorphous form, since it is oxidized and inactivated by moist air within a few days at room temperature. The second challenge is that low potency formulations require the addition of exceedingly small amounts of neat vitamin D which causes content uniformity issues. For example, a once-daily formulation containing the recommended daily intake of Vitamin D of 400 International Units (1 I.U.=0.025 microgram) would require the addition of only 10 micrograms per dosage unit (tablet, capsule, etc.).

### SUMMARY OF THE INVENTION

This invention is directed to a process for preparing a stabilized form of vitamin D. This process dramatically improves the stability of vitamin D with respect to that of the unformulated material and also allows for excellent content uniformity in very low vitamin D potency formulations.

### BRIEF DESCRIPTION OF THE DRAWINGS

FIG. 1 depicts the Chemical Stability of Neat, Crystalline Vitamin D at 40° C. and Various Humidities.

FIG. 2 depicts the Chemical Stability of Formulated Vitamin D at 40° C. and 40° C./75% RH.

### DETAILED DESCRIPTION OF THE INVENTION

An embodiment of the instant invention is a process for preparing a stabilized form of vitamin D which comprises the steps of:
a) dissolving vitamin D in a solution that contains water and at least one surfactant to produce a mixture;
b) spraying the mixture onto an inert carrier to produce a wet mass; and
c) drying the wet mass to obtain the stabilized form of Vitamin D.

In a second embodiment of this invention is a process for preparing a stabilized form of vitamin D which comprises the steps of:
a) dissolving vitamin D in a solution containing water, at least one surfactant and at least one antioxidant, to produce a mixture;
b) spraying the mixture onto a carrier to produce a wet mass; and
c) drying the wet mass to obtain the stabilized form of Vitamin D.

In a further embodiment of the instant invention, an anionic surfactant is utilized in step a.

A further embodiment of this invention is the process for preparing a stabilized form of vitamin D which comprises the steps of:
a) dissolving vitamin D in an alcohol;
b) adding the dissolved vitamin D to a solution containing water, at least one surfactant and at least one antioxidant to produce a mixture;
c) spraying the mixture onto a carrier to produce a wet mass; and
d) drying the wet mass to obtain the stabilized form of Vitamin D.

In a third embodiment of the instant invention, the process comprises the steps of:
a) dissolving vitamin D in ethanol containing phenolic antioxidants, to produce a vitamin D/antioxidant solution;
b) adding the vitamin D/antioxidant solution to a solution containing water and at least one surfactant to produce a mixture;
c) spraying the mixture onto microcrystalline cellulose to produce a wet mass; and
d) drying the wet mass to obtain the stabilized form of Vitamin D.

In a further embodiment, the surfactant is sodium lauryl sulfate.

In a further embodiment, the antioxidant is butylated hydroxytoluene (BHT), propyl gallate (PG), ethylenediaminotretraacetic acid (EDTA), or combinations thereof.

Adequate vitamin D intake is essential to facilitate intestinal absorption of calcium, plays a critical role in regulating calcium metabolism, and is critically important in the mineralization of the skeleton. The primary biological function of vitamin D is to maintain calcium homeostasis by increasing the intestine's efficiency in absorbing dietary calcium and thereby helping ensure that the amount of calcium absorbed is adequate to maintain blood calcium in the normal range and adequate to maintain skeletal mineralization.

As used herein, the term “Vitamin D” refers to Vitamin D₂ (ergocalciferol), vitamin D₃ (cholecalciferol) or 25-hydroxy-cholecalciferol, which are non-activated forms of vitamin D. In a specific embodiment, “Vitamin D” is vitamin D₃ (cholecalciferol). Vitamin D₃ is a precursor of the hydroxylated, biologically active metabolites and analogues of vitamin D₃, i.e. 1α-hydroxy-cholecalciferol, and 1α,25-dihydroxy-cholecalciferol. Generally cholecalciferol may be activated by hydroxylation into 25-hydroxy-cholecalciferol (a non-activated vitamin D₃ analogue), and 25-hydroxy-cholecalciferol may be further hydroxylated at the 1α-position to 1,25-dihydroxy-cholecalciferol (an active form of vitamin D₃). Vitamins D₂ and D₃ have similar biological efficacy in humans. Unlike 25-hydroxylated-vitamin D₃, a non-activated metabolite of vitamin D₃, “active vitamin D₃ analogs,” e.g. 1α-hydroxy-vitamin D₃ and 1α,25-dihydroxy-holecalciferol, cannot be administered in large dosages on an intermittent schedule due to their toxicity to mammals. However, 25-hydroxy-cholecalciferol, a non-activated vitamin D₃ metabolite and the primary storage form of vitamin D in the human body, may be administered in larger doses on an intermittent basis than “active” forms of vitamin D without toxicity. The intrinsic activity of 25-hydroxy-cholecalciferol is about 100 fold lower than that of 1α,25-dihydroxy-cholecalciferol. The phrase “intrinsic activity” may be defined as the ability of the vitamin D analog to act as an agonist at the level of the vitamin D receptor, without need for enzymatic activation by the 1α-hydroxylase enzyme, to either calcitriol itself (the natural hormone metabolite of vitamin D₃, also known as 1α,25-dihydroxy-cholecalciferol) or a chemically similar analog, e.g. 1α-hydroxy-cholecalciferol or dihydrotachysterol₂ which also do not require 1α-hydroxylation for activity. All other forms of vitamin D that require 1α-hydroxylation are considered non-activated, e.g. 24,25-dihydroxy-cholecalciferol, vitamin D₂, vitamin D₃, and 25-hydroxy-cholecalciferol. See Philip Felig, M. D. et al., Endocrinology & Metabolism, 4^{th} Edition, McGraw-Hill, Inc., Medical Publishing Division, pp. 1098-1109 (2001), which is incorporated herein in its entirety by reference thereto.

Vitamin D insufficiency can be age related, or due to geographical and seasonal causes. While exposure to sunlight provides most of the vitamin D required for children and young adults, the body can deplete its stored vitamin D because of a lack of exposure to sunlight combined with a dietary deficiency. Darkly pigmented skin and the skin of the elderly are believed to be less efficient in synthesizing vitamin D₃, especially during the winter months and in northern latitudes. Aging and renal impairment can also reduce the efficiency of vitamin D metabolism. To further compound this problem, through an independent mechanism, the efficiency of intestinal calcium absorption decreases with increasing age. Although vitamin D₃ can be derived from dietary sources, the amounts of constitutive vitamin D₃ in foods is low. To compensate for dietary deficiencies, some countries supplement certain foods, such as milk, margarine, cereals, and bread with vitamin D (Glenville, J., Pharmacological Mechanisms of Therapeutics: Vitamin D and Analogs, Principles of Bone Biology, 1069-1081 (1996)). However, vitamin D supplementation of food fails to ensure adequate intake, especially among the elderly who do not frequently consume these foods. As a result, vitamin D deficiency is particularly problematic in older people where intestinal absorption of calcium is less efficient, and dietary deficiencies and low sunlight exposure are common.

Vitamin D deficiency and vitamin D insufficiency remain neglected problems. In New England during the winter, it is estimated that 57% or more of inpatients and 40% of outpatients are vitamin D insufficient or deficient (Malabanan, A. et al., Redefining Vitamin D deficiency. Lancet 351, 805-806 (1998)). Approximately 30% of osteoporotic patients in the United States, European Union and Asia have some degree of vitamin D insufficiency which may be reversed with vitamin D supplementation. The prevalence of low 25-hydroxy vitamin D₃ metabolite levels in elderly long-term care patients approaches 100% in Northern Europe and in North America. The prevalence of 25-hydroxy vitamin D₃ insufficiency and deficiency in healthy elderly in Northern Europe is about 50% and 15%, respectively. In North America and Scandinavia, nearly 25% of the elderly women have winter 25-hydroxy vitamin D₃ levels that are below normal limits. Finally, according to studies conducted in Europe, the majority of elderly patients with hip fractures had 25-hydroxy vitamin D levels within the osteomalacia range. Two-thirds of hip fracture patients in Northern Europe have vitamin D₃ deficiency. The prevalence of vitamin D insufficiency and deficiency creates a medical need for vitamin D supplementation in the patient populations prone to, or suffering from, osteoporosis or osteopenia and in the subjects undergoing bisphosphonate therapy.

The process of the instant invention deposits a mixture of vitamin D and a surfactant onto the surface of a carrier, such as microcrystalline cellulose, which significantly improves the thermal stability of vitamin D as compared to bulk vitamin D. This process also effectively “dilutes” vitamin D onto the carrier, facilitating the addition of larger amounts of material that can be efficiently blended, resulting in excellent vitamin D content uniformity even for low vitamin D potency formulations.

The vitamin D mixture that is sprayed onto carrier may optionally include binders such as hydroxypropyl cellulose (HPC), hydroxypropyl methylcellulose (HPMC), polyvinylpyrrolidone (PVP) or starch 1500 to help maintain a strong bond between the vitamin D and the carrier.

For processing, the stabilized solution, with or without a binder, can be processed by high shear wet granulation, low shear wet granulation (both followed by a drying step) or fluid bed granulation onto a carrier or mixture of carriers. Prior to combination with other ingredients or another active ingredient to form a stabilized vitamin D-containing product, a super disintegrant sodium starch glycolate or croscarmellose sodium may be added to assist in granule disintegration.

As used herein, the term “surfactants” includes anionic surfactants, cationic surfactants and nonionic surfactants. Examples of anionic surfactants include, but are not limited to, alpha olefin sulfonate, ammonium laureth sulfate, ammonium laureth ether sulfate, ammonium stearate, sodium laureth sulfate, sodium lauryl sulfate, sodium octyl sulfate, sodium sulfosuccinimate, sodium tridecyl ether sulfate, triethanolamine lauryl sulfate, combinations thereof and the like. Examples of cationic surfactants include, but are not limited to, cetylpyridinium chloride, dimethylbenzylammonium chloride, benzalkonium chloride, lauryltrimethyl ammonium chloride, cetyltrimethylammonium chloride, stearyltrimethylammonium chloride, myristyltrimethylammonium chloride, and the like. Examples of non-ionic surfactants include, but are not limited to, various Tweens, poloxamers, Brijs, PEG-lated fatty acids, tocopherol polyethylenesuccinate ester, etc. In one embodiment, the surfactant is an anionic surfactant. In a further embodiment, the surfactant is sodium lauryl sulfate.

Examples of antioxidants include, but are not limited to, vitamin A, vitamin C, vitamin E (tocopherol), butylated hydroxytoluene (BHT), butylated hydroxyanisol (BHA), propyl gallate (PG), hydroquinone, α-tocopherol, ascorbic acid, ascorbyl palmitate, vitamin E palmitate, sodium bisulfite, ethylenediaminotretraacetic acid (EDTA), combinations thereof, and the like. In an embodiment, the antioxidant is ascorbyl palmitate, BHA, PG, BHT, EDTA, sodium bisulfite or combinations thereof. Specific phenolic antioxidants include, but are not limited to, butylated hydroxyanisol (BHA), propyl gallate (PG), butylated hydroxytoluene (B HT), vitamin E (tocopherol), hydroquinone or combinations thereof. In another embodiment, the antioxidant is butylated hydroxytoluene (BHT), propyl gallate (PG), ethylenediamino-tretraacetic acid (EDTA), or combinations thereof.

As used herein, the term “carrier” refers to an inert excipient suitable to perform as a support material and to perform as a “diluent” for the vitamin D/SLS mixture. Examples of a carrier include, but are not limited to, microcrystalline cellulose, lactose, mannitol, calcium phosphate, dicalcium phosphate and the like.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

### Example 1

A 15% sodium lauryl sulfate (SLS) solution was prepared by dissolving 75.022 g SLS in 500 ml of water. Concurrently, a stock solution containing vitamin D₃, butylated hydroxytoluene, and propyl gallate was prepared by dissolving 800.19 mg vitamin D₃, 801.37 mg BHT, and 801.01 mg PG in 10 ml absolute ethanol. Next, 5.25 g of disodium EDTA was weighed into a flask, and 150 ml of the 15% SLS solution was added. Once this material had dissolved, 3.75 ml of the vitamin D₃/antioxidant stock solution was delivered and the combined solution was stirred. Granulation was achieved by spraying this solution onto 256 g of Avicel PH102 in a Bohle mini-granulator (BMG), at a spray rate of 25 ml/min. The contents of the BMG were removed, dried overnight in a 40° C. vacuum oven, and sieved through a 355 μm mesh. Those materials passing through the screen were collected.

Stability Data

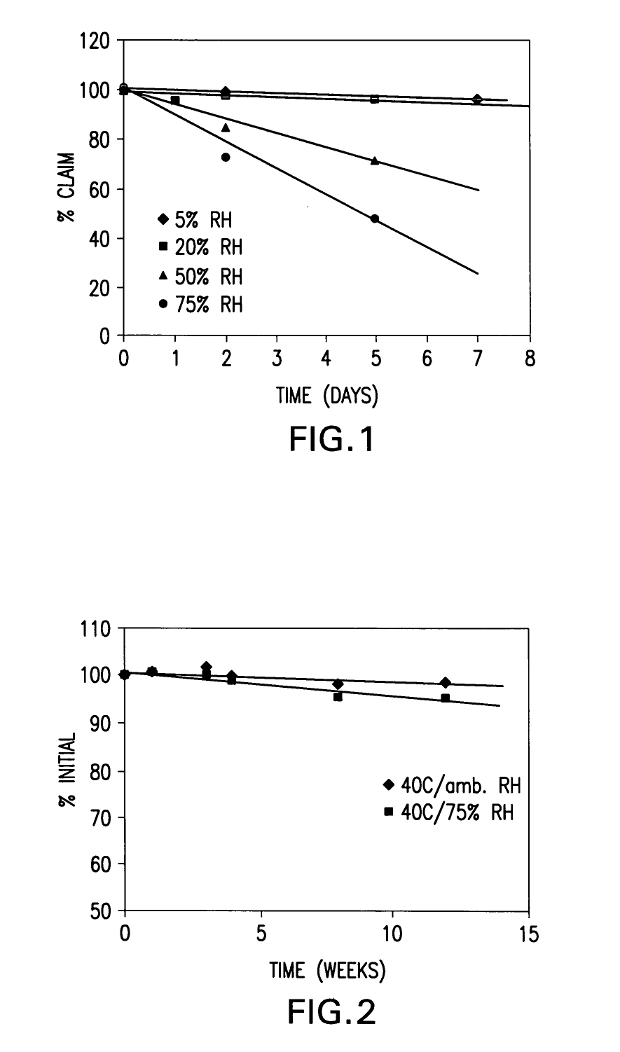

Stability of neat, crystalline vitamin D₃ was investigated at 40° C. and 5%, 20%, 50%, 75% relative humidity (% RH) over the course of one week. This experiment was used as a baseline for comparison of vitamin D₃ stability after formulation as indicated above. A pronounced dependence of degradation rate upon relative humidity was observed, as depicted in FIG. 1. Under low humidity conditions, loss of active occurred at a rate of 0.58%/day and 0.76%/day at 5% RH and 20% RH, respectively. At 50% RH and 75% RH, even more severe degradation was observed at a rate of 5.5%/day and 10.8%/day, respectively.

In an additional experiment, vitamin D₃ was cast as a thin film by evaporating a solution of vitamin D in ethyl acetate and the stability of this amorphous form of vitamin D was also investigated. Essentially complete degradation was observed after 3 days at 25° C., 40° C., and 40° C./75% RH. The results of this and the previous experiment highlight the inherently low stability of vitamin D and the impact of both humidity and physical form (crystalline vs. amorphous) on degradation rates.

The stability of vitamin D₃ in the formulation described in Example 1 was also investigated at 40° C./ambient humidity, and 40° C./75% RH, with respect to −20° C. control samples. The results showed that formulating vitamin D in the manner described dramatically reduced the rate of degradation of vitamin D₃ (FIG. 2). At 40° C./amb. RH, the rate of vitamin D₃ loss was only 0.24%/week as compared to 0.24%/day-5.5%/day in the range 20% RH-50% RH for the unformulated crystalline material. This represents a stability enhancement of 22- to 160-fold. At 40° C./75% RH, the observed degradation rate was 0.50%/week suggesting that under these conditions, vitamin D₃ shelf life could be effectively extended by >150-fold by means of this simple formulation. No detectable degradation was observed in the −20° C. control samples during the 12 weeks of the experiment.

What is claimed is:

1. A process for preparing a stabilized form of vitamin D which comprises the steps of:
a) dissolving vitamin D in a solution that contains water and at least one surfactant to produce a mixture;
b) spraying the mixture onto an inert carrier to produce a wet mass; and
c) drying the wet mass to obtain the stabilized form of vitamin D.

2. The process of claim 1, which further comprises the dissolving of vitamin D in a solution containing water, at least one surfactant and at least one antioxidant, to produce a mixture in step a).

3. The process of claim 2, wherein an anionic surfactant is utilized in step a.

4. The process of claim 2, wherein the surfactant is selected from sodium lauryl sulfate, ascorbic acid, ascorbic acid palmitate, sodium bisulfite, ethylenediaminotetraacetic acid (EDTA), or combinations thereof.

5. The process of claim 4, wherein the surfactant is sodium lauryl sulfate.

6. A process for preparing a stabilized form of vitamin D which comprises the steps of:
a) dissolving vitamin D in an alcohol;
b) adding the dissolved vitamin D to a solution containing water, at least one surfactant and at least one antioxidant to produce a mixture;
c) spraying the mixture onto a carier to produce a wet mass; and
d) drying the wet mass obtain the stabilized form of vitamin D.

7. The process of claim 6, wherein the antioxidant is a phenolic antioxidant.

8. The process of claim 7, wherein the antioxidant is butylated hydroxyanisol (BHA), propyl gallate (PG), butylated hydroxytoluene (BHT), or combinations thereof.

9. A process for preparing a stabilized form of vitamin D₃ which comprises the steps of:
a) dissolving vitamin D₃ in ethanol containing phenolic antioxidants, to produce a vitamin D₃/antioxidant solution;
b) adding the vitamin D₃/antioxidant solution to a solution containing water and at least one surfactant to produce a mixture;
c) spraying the mixture onto microcrystalline cellulose to produce a wet mass; and
d) drying the wet mass obtain the stabilized form of vitamin D₃.

10. The process of claim 8, wherein the antioxidant is butylated hydroxyanisol (BHA), propyl gallate (PG), butylated hydroxytoluene (BHT), or combinations thereof.

11. The process of claim 9, wherein the surfactant is selected from sodium lauryl sulfate, ascorbic acid, ascorbic acid palmitate, sodium bisulfite, ethylenediaminotetraacetic acid (EDTA), or combinations thereof.

12. The process of claim 11, wherein the surfactant is sodium lauryl sulfate.

13. The process of claim 12, wherein the surfactant is sodium lauryl sulfate with ascorbic acid.
